(19) United States
(12) Patent Application Publication
(10) Pub. No.: US 2009/0120441 A1
(43) Pub. Date: May 14, 2009

(54) Nasal cavity filter and manufacturing method thereof

(76) Inventor: Wen-kuang Wang, Taichung (TW)

Correspondence Address: SAM CHEN, 7F-1, 293, ROOSEVELT ROAD, SEC 3, TAIPEI, TW

(21) Appl. No.: 11/938,792

(22) Filed: Nov. 13, 2007

(51) Int. Cl. A61M 16/00

(52) U.S. Cl. 128/206.11; 425/192 R

(57) ABSTRACT

A nasal cavity filter includes two horn-shaped filter units each having a filter layer fixed therein. The shape of the filter layer is disposed relative to that of the filter unit. The filter unit is made of flexible material and the filter layer is made of soft material. The two filter units are respectively inserted into two nasal passages of user's nose and connected by an arc bridge that goes across a distal end of user's nasal bone.

### BACKGROUND OF THE INVENTION

1. Field of the Invention

The present invention relates to a filter and manufacturing method thereof, and more particularly to a nasal cavity filter that is received in two nasal passages of the user.

2. Description of Related Art

Allergic rhinitis or pollenosis is a familiar disease in nasal outpatient services. The best and the cheapest way for preventing human from allergic rhinitis or pollenosis is to wear the gauze mask. The gauze mask can filter off some inhalant allergens. However, to wear gauze mask has some inconveniences and disadvantages as followed.

1. Bringing muggy feeling: the gauze mask covers the user's mouth and nose such that the user easily feels muggy, especially in a hot climate or an operating place that has a high temperature.

2. It is weak for personal hygiene. The gauze mask covers user's mouth and nose during wearing such that the germs from user's mouth and nose will be cohered and reproduced on the lining of the gauze mask. As well known, most of the users do not abandon the gauze mask only used for one time. The user may infect with the germs from the used gauze mask.

3. The gauze mask can not fully abut against user's face. The gauze mask usually is positioned by using two elastic straps that surrounds user's ears. However, the pull force from the two elastic straps will change the shape of the gauze mask such that the gauze mask can not fully abut against user's face and the filter effect is reduced.

Furthermore, some specific persons can not use gauze mask during work, such as a traffic police. The traffic police needs to blow a whistle when ordering traffic such that he/she can not use gauze mask because the gauze mask covers his/her mouth. In addition, the air pollution from the driving cars is serious in the crossroads. The traffic police need an improved nasal filter to protect his/her lung from polluted air.

The present invention has arisen to mitigate and/or obviate the disadvantages of the conventional gauze mask.

### SUMMARY OF THE INVENTION

The main objective of the present invention is to provide an improved nasal cavity filter that is inserted into user's nostril during using.

To achieve the objective, the nasal cavity filter in accordance with the present invention comprises two horn-shaped filter units each having a filter layer fixed therein. The shape of the filter layer is disposed relative to that of the filter unit. The filter unit is made of flexible material and the filter layer is made of soft material. The two filter units are respectively inserted into two nasal passages of user's nose and connected by an arc bridge that goes across a distal end of user's nasal bone.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a nasal cavity filter in accordance with the present invention;

FIG. 2 is an exploded perspective view of the nasal cavity filter in FIG. 1;

FIG. 3 is a cross-sectional view of the nasal cavity filter in FIG. 1 along line A-A;

FIG. 3A is a partially enlarged view of FIG. 3; and

FIG. 4 is a schematic view of the nasal cavity filter in accordance with the present invention.

### DETAILED DESCRIPTION OF THE INVENTION

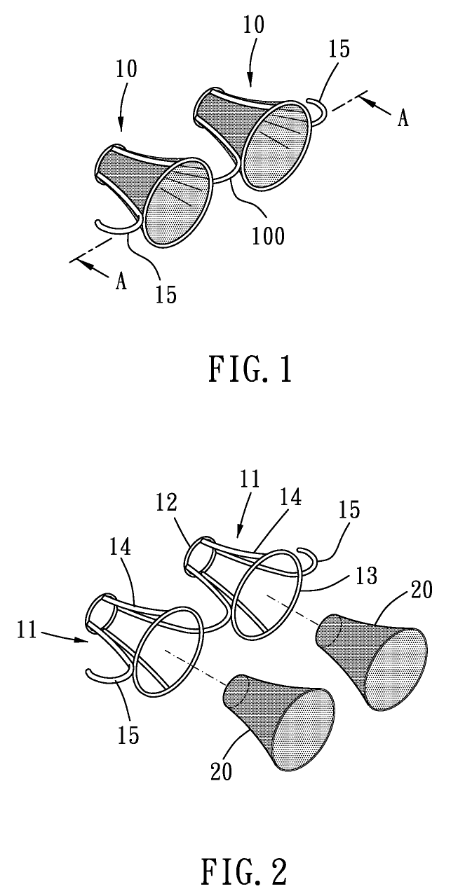

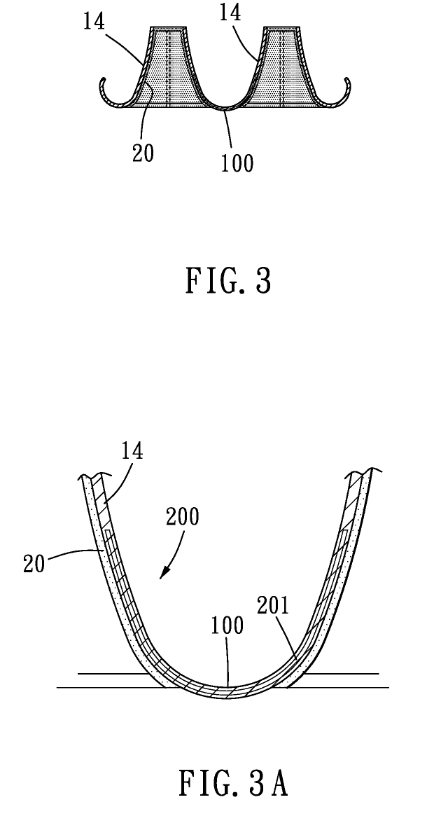

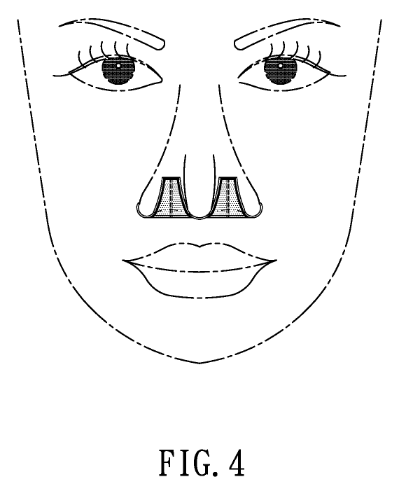

Referring to the drawings and initially to FIGS. 1-4, a nasal cavity filter in accordance with the present invention comprises two horn-shaped filter units (10) each having a filter layer (20) fixed therein. The shape of the filter layer (20) is disposed relative to that of the filter unit (10). The filter unit (10) is made of flexible material and the filter layer (20) is made of soft material. The two filter units (10) are respectively inserted into two nasal passages of user's nose and connected by an arc bridge (100) that goes across a distal end of user's nasal bone.

The filter unit (10) comprises a framework (11) including a first ring (12), a second ring (13) and multiple ribs (14) to connect the first ring (12) and the second ring (13) for forming a horn-shaped structure. In the preferred embodiment of the present invention, the filter unit (10) has four ribs (14), as shown in FIG. 2. One of the ribs (14) of each of the two filter units (10) is longitudinally connected to a corresponding one of two opposite ends of the bridge (100). The bridge (100) and the two ribs (14), which are respectively connected to the two opposite ends of the bridges (100) integrally form a U-shaped structure (200) for clamping the nasal bone to hold the nasal cavity filter in place. A plasticized wicker (201) is received in the U-shaped structure (200) for adjusting the shape of the U-shaped structure (200) and promoting the comfort during wearing the nasal cavity filter in accordance with the present invention. One of the ribs (14) of each of the filter (10), opposite to the bridge (100) has a hook (15) outward extending therefrom. The hooks (15) are provided to hook user's nasal alae for promoting the stability of the nasal cavity filter of the present invention when being worn.

The size of the present invention is limited to prevent the filter unit (10) from contacting with the vibrissa to cause a sense of titillation or sneezing. The second ring (13) is designed relative to the shape and the size of human's nostril. In addition, the framework (11) is made of flexible material such that the second ring (13) can be slightly compressed for a small nostril. Otherwise, the second ring (13) can be made with various sizes or a child of a person who has a big nostril. The first ring (12) has a third area relative to that of the second ring (13) and the vertical distance between the first ring (12) and the second ring (13) is installed about one centimeter to prevent the filter unit (10) from overly extending into the nasal passage of user's nose.

Regarding to the manufacturing method of the nasal cavity filter in accordance with the present invention, two filter layers (20) and a plasticized wicker (201) are previously shaped to two horn-shaped structures and a U-shaped structure. The shaped filter layers (20) and the plasticized wicker (201) are previously positioned in the mold such that the framework (11) and the bridge (100) are respectively formed with the two filter layers (20) and the plasticized wicker (201) in the mold by injection molding. The framework (11) and the bridge (100) are formed by transparent material or skin tone material for being pleasing to the eye during wearing.

As described above, the nasal cavity filter in accordance with the present invention has several advantages as follow.

1. It is comfortable during wearing. The filter unit (10) and the filter layer (20) of the present invention are respectively made of flexible material and soft material such that the U-shaped structure (200) should not overly clamp the nasal bone of the user and the plasticized wicker (201) can adjust the width of the U-shaped structure (200) for being suitable to various users.

2. It is easy to manufacture. The nasal cavity filter of the present invention is completely formed in the mold such that the nasal cavity filter of the present invention has a simple manufacturing process and a low price. Consequently, the nasal cavity filter of the present invention is abandoned after being used for one time without washing.

3. It is convenient for wearing. The nasal cavity filter is inserted into the user's nostrils without covering the user's mouth. Consequently, the user can freely use his/her mouth to eat or blow whistle.

4. The framework (11) and the bridge (100) are formed by transparent material or skin tone material that are pleasing to the eye during wearing.

Although the invention has been explained in relation to its preferred embodiment, it is to be understood that many other possible modifications and variations can be made without departing from the spirit and scope of the invention as hereinafter claimed.

What is claimed is:

1. A nasal cavity filter comprising two horn-shaped filter units each having a filter layer fixed therein and shape of the filter layer being disposed relative to that of the filter unit, wherein the filter unit is made of flexible material and the filter layer is made of soft material, the two filter units respectively inserted into two nasal passages of user's nose and connected by an arc bridge that goes across a distal end of user's nasal bone, each filter unit comprising a framework including a first ring, a second ring and multiple ribs to connect the first ring and the second ring for forming a horn-shaped structure, one of the ribs of each of the two filter units is longitudinally connected to a corresponding one of two opposite ends of the bridge, the bridge and the two ribs, which are respectively connected to the two opposite ends of the bridges integrally form a U-shaped structure for clamping the nasal bone to hold the nasal cavity filter in place.

2. The nasal cavity filter as claimed in claim 1 further comprising a plasticized wicker received in the U-shaped structure for adjusting the shape of the U-shaped structure and promoting the comfort during wearing.

3. The nasal cavity filter as claimed in claim 1, wherein one of the ribs of each of the filter, opposite to the bridge has a hook outward extending therefrom and hooking user's nasal alae for promoting the stability of the nasal cavity filter during wearing.

4. The nasal cavity filter as claimed in claim 1, wherein the first ring has a third area relative to that of the second ring to prevent the filter unit from contacting with the vibrissa to cause a sense of titillation or sneezing.

5. The nasal cavity filter as claimed in claim 2, wherein one of the ribs of each of the filter, opposite to the bridge has a hook outward extending therefrom and hooking user's nasal alae for promoting the stability of the nasal cavity filter during wearing.

6. The nasal cavity filter as claimed in claim 2, wherein the first ring has a third area relative to that of the second ring to prevent the filter unit from contacting with the vibrissa to cause a sense of titillation or sneezing.

7. The nasal cavity filter as claimed in claim 3, wherein the first ring has a third area relative to that of the second ring to prevent the filter unit from contacting with the vibrissa to cause a sense of titillation or sneezing.

8. A method for manufacturing a nasal cavity filter comprising the steps of:
shaping two filter layers into two horn-shaped structures and shaping a plasticized wicker into a U-shaped structure respectively;
positioning the shaped filter layers and the plasticized wicker in the mold; and
forming a framework with the two filter layers in the mold by injection molding and forming a bridge with the plasticized wicker in the mold by injection molding respectively.

9. The method as claimed in claim 8, wherein the framework is formed a first ring, a second ring and multiple ribs to connect the first ring and the second ring.

10. The method as claimed in claim 8, wherein the framework and the bridge are made of transparent material for being pleasing to the eye during wearing.

11. The method as claimed in claim 8, wherein the framework and the bridge are made of skin tone material for being pleasing to the eye during wearing.

12. The method as claimed in claim 9, wherein the framework and the bridge are made of transparent material for being pleasing to the eye during wearing.

13. The method as claimed in claim 9, wherein the framework and the bridge are made of skin tone material for being pleasing to the eye during wearing.
